(19) United States
(12) Patent Application Publication
(10) Pub. No.: US 2006/0167383 A1
(43) Pub. Date: Jul. 27, 2006

(54) Screening methods and kits for gastrointestinal diseases

(76) Inventors: Maciej J. Kieturakis, Redwood City, CA (US); Paul Czubarow, Wellesley, MA (US); Maciej Patelka, Brighton, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/391,068

(22) Filed: Mar. 27, 2006

Related U.S. Application Data
(63) Continuation-in-part of application No. 10/405,925, filed on Apr. 1, 2003.

(51) Int. Cl. A61B 5/00

(52) U.S. Cl. 600/584

(57) ABSTRACT

Methods and systems for detecting occult blood and other analytes in the water of a toilet bowl release a dye reagent into the water which produces an observable signal in the presence of the blood or other selected analytes. The dye reagent is preferably dispersed as a liquid, powder, gel, or other form which rapidly mixes and combines with the sample. Additionally, the water of a toilet bowl will have a reduced impurity content such as iron and a surfactant is used to help liberate the analyte from a stool sample. Usually, automatic mechanical or electromechanical dispensing systems are used to release the dye reagent and surfactant into the water.

### CROSS-REFERENCES TO RELATED APPLICATIONS

This application is a continuation-in-part of U.S. patent application Ser. No. 10/405,925 (Attorney Docket No. 017575-000600US), filed on Apr. 1, 2003, the full disclosure of which is incorporated herein by reference.

### BACKGROUND OF THE INVENTION

1. Field of the Invention

The present invention relates generally to medical diagnostic methods and systems. More particularly, the present invention relates to methods and systems for detecting occult blood or other analytes in the water of the bowl of a flush toilet.

Colon and rectal cancers are a leading cause of death and disability throughout the world. Early detection and treatment of both diseases can significantly increase the chances of a patient's survival. A common diagnostic test for both diseases relies on the detection of occult blood in patient feces. Occult blood detection is most commonly performed by the patient obtaining small stool samples, spreading those samples thinly over a specially treated substrate, allowing the substrates to dry, and sending the dried substrates to a central laboratory or a doctor's office for testing. Usually, repeat samples will be taken over several days and collected prior to sending them for laboratory evaluation.

While quite useful if performed correctly, such home diagnostic stool testing suffers from both poor compliance and incompetent performance. Most patients are quite reluctant to process stool samples, even their own. Because of this reluctance, and undoubtedly for other reasons as well, many patients are unable to properly collect the samples, apply them to the substrates, and maintain the samples in proper condition before they are sent to the testing laboratory. Because of these problems, many patients who had been advised to sample their stool never complete the home testing program, and many of those tests which are completed are compromised so that the test reliability is reduced. Additionally, many patients are reluctant to visit their physician for periodic exams and screening tests, therefore such fecal occult blood detection tests are never even initiated.

To promote compliance and reduce complexity, performance of occult blood assays directly in the water bowl of a toilet has been proposed. A variety of tablets, solid phase substrates, and other diagnostic agents have been formulated, where the user can simply drop these agents into the toilet bowl after use. While theoretically increasing patient compliance, the patient can still make mistakes in adding the reagents. The addition of dried reagents and related carriers can present mixing problems which limit the accuracy of the test. Moreover, the completion of such testing requires patient compliance, which is frequently absent due to a variety of factors.

Testing of liquid samples such as urine is usually easier than testing stool samples. This is because the urine is an aqueous solution of chemicals that is easily delivered to the toilet. Analytes in the urine dissolve and disperse easily in the water of the toilet bowl and the volume of urine is a large proportion of the total volume of water in the toilet bowl. Thus, there is a relatively large concentration of analyte dispersed in the water of the toilet bowl. This makes analyte detection fairly easy, and thus impurities, natural salts, or other ions such as iron, in the toilet bowl water do not interfere with detection of readily available hemoglobin or other analytes.

Testing stool samples is however, more difficult since the analyte is trapped in the solid stool sample and hence only minute quantities of analyte are released from the surface of the stool into the toilet bowl water. In this case, there may be a relatively low concentration of analyte in the toilet bowl water, so impurities, natural salts or ions such as iron in the water can interfere with the assay. Therefore, it is desirable to improve the assay test conditions by reducing impurities such as iron from the water.

Filters may be used to remove some impurities while ion exchange resins are well known in the field and are commonly used as water softeners to remove other impurities. In the typical ion exchange resin system, untreated water is passed through a reservoir filled with small polystyrene beads, also known as ion exchange resin. These beads carry a negative charge and are covered with sodium ions. As the untreated water is passed through the resin, the negatively charged beads attract positively charged ions in the water. The sodium on the resin is then replaced or swapped with natural salts such as calcium, magnesium and iron from the water, thereby producing treated water with a reduced ion content. This water allows analyte assays to be performed in the toilet bowl without interference from contaminants and thus provides a better environment for the assay. Eventually, the resin becomes saturated with ions and must be regenerated by passing a stream of sodium solution over the beads to wash away and displace all of the ions that have built up on the resin. Some of the sodium remains in the treated water, but this does not hinder the assay.

The use of surfactants also helps when testing stool samples. Stool is composed of fat-like substances and therefore the use of a surfactant, which is a surface acting compound, helps to break the external layers of the solid stool sample thereby releasing greater amounts of analyte into the toilet bowl water which increases the likelihood of detection during the assay. The surfactant may also help in the dissolution of the dye used during testing.

For these reasons, it would be desirable to provide improved methods, systems, and reagents for performing occult blood testing in situ in the water bowl of a flush toilet. It would be further desirable to provide such methods and systems which are also useful for detecting other analytes in an analogous manner. The methods and systems should further reduce or eliminate the level of skill required by the patient to perform the assay. It would be particularly desirable if such methods and systems were to proceed automatically each time a toilet is used for defecation or urination, either by responding to the flushing of the toilet or to the use of the toilet in other ways. Such methods and systems would desirably further provide for unambiguous results and permit easy reading of those results by the patient. Additionally, the methods and systems should facilitate the release of the analyte from stool samples as well as minimizing impurities including iron and other ionic contaminants in the toilet water that could interfere with assay results. At least some of these objectives will be met by the inventions described herein below.

2. Description of the Background Art

The preparation of dried reagents which may be added to stool in a toilet to perform occult blood assays is described in U.S. Pat. No. 4,956,300. Other patents of interest include U.S. Pat. Nos. 6,271,046 B1; 6,221,678 B1; 6,186,946 B1; 5,196,167; 5,192,501; 5,081,040; 4,725,553; 4,625,160; 4,672,654; 4,541,987; 4,511,533; 4,175,923; and 2,828,377. Toilets which are capable of performing many functions, including measuring blood in urine, are predicted in “Japanese Masters Get Closer to the Toilet Nirvana,” New York Times, Oct. 8, 2002.

### BRIEF SUMMARY OF THE INVENTION

According to the present invention, occult blood and other analytes symptomatic of gastrointestinal disease are detected by the addition of a dye reagent and surfactant directly into the water of the bowl of a flush toilet. A sufficient quantity of surfactant is preferably used to help release analyte from the test sample. The dye reagent reacts with the analyte, if present, to produce an observable signal, usually a color change in the water.

The water preferably has a reduced impurity content and/or iron content because iron can react with the dye reagent to produce false test results for occult blood. Similarly, other impurities that might affect detection of other analytes should also be reduced from the water. Reduced impurity and iron content in the water are typically achieved with filters and/or an ion exchange resin. Exemplary resins include Dowex G-26, Dowex 21K XLT, Dowex MAC-3 and MTO-Amberlite, all manufactured by Supelco. Other means for reducing iron content are available, such as an electrolytic ion exchange system. The water may be treated to remove impurities and iron before being released into the toilet bowl or before release into a water tank of the flush toilet.

In a first aspect of the present invention, the dye reagent and surfactant are dispersed into the water, typically being in a liquid, gel, powder, or solid form which rapidly dissolves in the water and mixes with a stool sample to promote accurate and immediate results. In a second aspect of the present invention, the dye reagent and surfactant are dispensed into the toilet bowl water in response to use of the toilet, such as flushing, sitting, or by the selective manual activation of a dosing unit. In the third aspect of the present invention, systems are provided for automatically dispensing the dye reagents and surfactant into the toilet bowl water in response to use. In all aspects, a second detector or dye reagent may also be dispersed to produce a signal in the presence of a second analyte.

Dispersing or otherwise adding an amount of a dye reagent and surfactant to the toilet bowl water according to the first aspect of the present invention requires that the reagents be in a dispersible and/or soluble form, usually being solution (liquid), gel, powder, or solid form which rapidly dissolves and/or dispenses in the toilet bowl water. Such dispersible forms generally exclude tablets, solid phase substrates, and other forms which will not rapidly mix or dissolve with the toilet bowl water and with the stool sample therein. Usually, although not necessarily, such dispersible dye reagents and surfactant will be dispensed automatically in response to a use of the toilet, as will be described in more detail hereinbelow. Less preferably, however, the dispersible forms of the dye reagent and surfactant may also be selectively or manually released into the toilet bowl water, where they will quickly mix and react with the stool, producing an observable signal when the analyte is present.

The automatic dispensing of a dye reagent and surfactant, according to the second aspect of the present invention, will include the release of both dispersible and non-dispersible forms of the dye reagent and surfactant. That is, in addition to the liquid, gel, and powder forms of the dried reagent, the present invention further comprises automatically dispensing even non-dispersible forms, such as tablets, substrates, solid phases, and the like. Such automatic release may be in response to any use of the toilet, including flushing, sitting on the seat of the toilet, electronic proximity sensing of a patient using the toilet, detection of fecal matter entering the toilet bowl water, detecting a change in water level or turbulence in the water of the toilet bowl after any use, including vomiting, and the like. The latter relative use detection is particularly advantageous since it avoids the dispensing of reagent when the toilet is used without fecal matter entering the toilet bowl.

In all aspects of the methods of the present invention, the dye reagent and surfactant may be dispensed into the water in the toilet tank, directly into the water in the toilet bowl, or as some combination of both. For example, when the dye reagent comprises both a dye and a separate oxidizer, as described in more detail below, the dye and the oxidizer may be dispersed together or separately into the water, with either or both going into the water in the toilet tank or into the water in the toilet bowl. Similarly, the dye may be maintained with an anti-oxidant and/or a surfactant and dispersed into the water of a toilet tank or toilet bowl, as previously described. If the dye is maintained with an anti-oxidant, then the oxidizer is preferably maintained separately from the dye.

The methods of the present invention preferably provide for dispensing or releasing measured amounts of the dye reagent and surfactant into the tank, typically in response to flushing. Usually, such dispensing comprises dropping a measured amount of a liquid, gel, or powder. In other instances, however, dispensing may comprise dissolving an amount of a solid dye reagent (or reagent component) and solid surfactant into the tank or the bowl of the toilet.

In all instances, the presence of the dye reagent in the toilet bowl water will produce an observable signal in the presence of blood or other analyte, typically producing a color change in the presence of blood in the water of the toilet bowl. Preferably, the dye reagent will be selected and provided in an amount which produces an observable color change at a local blood concentration in the water of 0.2 ppm and above, preferably 0.1 ppm and above. Exemplary reagents comprise an oxidizer and a dye, where the oxidizer oxidizes the dye to produce a color change in the presence of a catalyst-peroxidase from blood hemoglobin. Exemplary dyes include 3,3′5,5′-tetramethylbenzidine, gum guaiac, potassium guaicosulfonate; phenolphthalin, 3,3′-dimethylbenzidine, o-toluidine, 4,4′-diaminobiphenyl, and the like. Exemplary oxidizers include alkali metal perborates, OXONE, hydrogen peroxide, and the like. Immunochromatographic detection employing a monoelonaldehyde conjugal with polyclonal antibodies might also be possible.

Optionally, the methods of the present invention may further comprise selectively adding a control reagent to the toilet bowl water to confirm that the system is working and optionally calibrate the system.

The methods of the present invention employ a surfactant in an amount sufficient to release analyte from stool. Usually this amount is in the range of 0.25% to 5% by weight (based on the weight of water present in the bowl and/or the weight of water introduced into the bowl by flushing) but may be up to 50% by weight. The surfactant typically comprises one or more surfactant selected from the group consisting of fatty alkanolamides, isopropyl amine branched dodecylbenzene sulfonates, calcium alkylbenzene sulfonates, hydrogenated cocamidopropyl betaines, anionic/lignosulfonates blends, linear alkylbenzene sulfonic acids, linear sodium dodecylbenzene sulfonates, and mixtures thereof.

Systems according to the third aspect of the present invention provide for automatically dispensing a dye reagent and surfactant into water in a bowl of a flush toilet. The systems include a reservoir holding an anti-oxidant and a dye reagent which is capable of producing an observable signal in the presence of analyte in the toilet bowl water, and a mechanism for dispensing an amount, preferably a measured amount, of the dye reagent into the water in the toilet bowl in response to a use of the toilet. The reservoir may also hold the surfactant, and/or the surfactant may be held in a second reservoir. Both dispensing mechanisms may be configured to release the dye reagent and surfactant in the water in the toilet bowl, or the water in the water tank, or some combination thereof, so that the water, dye reagent and surfactant are mixed in the toilet bowl. The dispensing mechanism may comprise a mechanical device which detects flushing of the toilet and/or rise of water in the toilet bowl or toilet tank and which releases a pre-measured amount of the dye reagent and surfactant in response to such detection. The pre-measured amount may be in the form of a liquid, gel, powder, or optionally be in a solid form, such as a tablet, solid phase substrate, or the like. Alternatively, the dispensing mechanism may dispense dye, surfactant and oxidizer separately into the water, where the dye, surfactant and/or oxidizer may be in any of the forms just mentioned. The dye reagent will be selected to provide an observable signal, the presence of the analyte typically producing a color change in the water in the presence of the analyte, such as blood. The system further comprises a means for reducing impurity content in the water of the toilet bowl, including iron. Typically, filters and an ion exchange resin are used to remove the iron from the water. Other electrolytic ion exchange methods may also be used to reduce impurity and iron content.

In the case of blood detection, the preferred detection ranges, reagent dye systems, surfactants, resins and the like, have been set forth above. The systems of the present invention may further comprise a control substance which may be added to the toilet bowl water to produce the observable signal when added to the water in the absence of the analyte.

### BRIEF DESCRIPTION OF THE DRAWINGS

FIG. 1 is a schematic illustration of a system according to the present invention comprising a dye dispensing or dispersing apparatus in the toilet tank of a toilet.

FIG. 2 is a schematic illustration of an alternative system according to the present invention wherein the dispensing or dispersing apparatus is located in the toilet bowl of the toilet.

FIG. 3 is a schematic illustration of a third embodiment of the system of the present invention comprising an electronic toilet use detector which can be arranged to control dispensing or dispersing of the dye reagents into either the toilet bowl or the toilet tank, or both.

FIG. 4 is a schematic illustration of another embodiment of the system of the present invention comprising a dye and surfactant dispensing or dispersing apparatus in the toilet tank of a toilet along with an ion exchange filter system for removing impurities and iron from the water of a toilet bowl.

FIG. 5 is a schematic illustration of an alternative system according to the present invention comprising an ion exchange filter system for reducing impurities and iron content from the water of a toilet bowl and wherein the dispensing or dispersing apparatus is located in the toilet bowl of the toilet.

FIG. 6 is a schematic illustration of a another embodiment of the system of the present invention comprising an electronic toilet use detector which can be arranged to control dispensing or dispersing of the dye reagents and surfactant into either the toilet bowl or the toilet tank, or both, and an ion exchange filter system for reducing impurities and iron content in the water of a toilet bowl.

### DETAILED DESCRIPTION OF THE INVENTION

Methods and systems according to the present invention will most commonly be used to detect fecal occult blood in stool samples in the bowl of a toilet. Such tests are useful for the early detection of colon cancer, rectal cancer, and other cancers of the gastrointestinal tract. They are also useful in detection of non-malignant gastrointestinal diseases such as gastritis, peptic ulcers and inflammatory bowel disease, which may otherwise persist undetected for a prolonged period of time. While the tests of the present invention will not be finally determinative of disease status, they will be very useful in alerting patients of the need to contact their physicians and have further testing done. In addition to the detection of fecal occult blood, the methods and systems of the present invention will also be useful to detect other analytes associated with diseases causing appearance of certain substances in the gastrointestinal tract like increased level of bilirubin in certain blood disorders, porphyrins in porhyrias, specific microorganisms in gastrointestinal infections, increased fecal fat levels in pancreatic exocrine insufficiency and others. Changes in the urine chemistry may detect diabetes (increased sugar level), renal insufficiency (proteins), renal malignancies or stones (urinary blood), increased urine calcium in parathyroidism, cathecholamines in pheochromocytoma, urine free cortisol in Cushing's disease and others. These chemical changes may also serve as a means for determining the desired serum levels of a variety of drugs used for other illnesses, or as a method of determining the day of ovulation to enhance conception efforts or as an early pregnancy test.

A particular advantage of the present invention is that it provides for automatic and daily screening of a patient's condition. As mentioned before, the screening will not be determinative of disease status, but will allow the patient to seek further diagnosis. For example, in the case of suspected colon or rectal cancer, subsequent screening by testing the stool for occult blood at the physician's office, colonoscopy or sigmoidoscopy would likely be in order.

The dye reagents useful in the present invention may take a variety of forms. Most simply, the reagent can be in the form of a dissolvable block which is placed in the toilet bowl or toilet tank so that it is exposed to water each time the water in the bowl or tank is replenished. Upon exposure to water, a pre-determined portion of the block will dissolve and release the dye reagent into the water. Such systems are commonly available for releasing cleaning and disinfecting reagents into toilets. The surfactant may take a similar form.

Alternatively, the dye reagent and surfactant may be released into the toilet bowl and/or toilet tank using a mechanical system which dispenses a pre-measured amount of the reagent in response to a use of the toilet, such as flushing, sitting, or the like. In the case of flushing, the mechanical linkage can be made directly to the handle or valve mechanism which initiates the flush, or it can be indirectly made to a response in the change of water level in the toilet bowl or tank. Such mechanical systems may release pre-measured amounts of the liquid, gel, powder, or other dispersible forms of the dry reagent or surfactant. Alternatively, the mechanical systems can release single or known numbers of tablet(s) upon each use of the toilet.

The present invention can further utilize electromechanical systems where various system components can be powered or motorized to enhance response. Additionally, the electromechanical systems can have electronic sensors incorporated for detecting a variety of events suitable for controlling the release of the dye reagents and surfactant. For example, sensors can sense the physical presence of a user, the positioning of the user on the toilet seat, release of fecal matter and/or urine into the toilet bowl water, or the like. Sensing these various events can be used to control the release of the dye reagent and surfactant using mechanical or electromechanical release means.

Generally, in the present invention, a reagent will be added to toilet water. A component of the reagent will react or bind with an analyte giving a characteristic and specific system change when the analyte is present. The change may be a color change or change of electric potential of toilet bowl solution specific for the analyte. Preferred dye reagent according to the present invention will comprise a dye and an oxidizer, wherein the oxidizer oxidizes the dye to produce a color released in the water in the presence of the analyte which acts as a catalyst. Exemplary dyes include 3,3′,5,5′-tetramethylbenzidine, gum guaiac, 3,3′-dimethylbenzidine, o-toluidine, 4,4′-diaminobiphen, and the like. A particularly preferred system is the combination of 3,3′,5,5′-tetramethylbenzidine and OXONE which reacts to produce blue dye in the presence of hemoglobin.

In addition to the reagents discussed above, surface active compounds may be used to help break external or deeper layers of solid stool samples. Surfactants are one example of such compounds that help to release greater amounts of the analyte (e.g. hemoglobin) from the test sample (e.g. stool) into the toilet bowl water, increasing the chance of detection during the assay. The concentration of surfactants used is typically in the range from 0.25% to 5% by weight, although up to 50% by weight may be used. Exemplary surfactants include fatty alkanolamides, isopropyl amine branched dodecylbenzene sulfonates, calcium alkylbenzene sulfonates, hydrogenated cocamidopropyl betaines, anionic/lignosulfonates blends, linear alkylbenzene sulfonic acids, linear sodium dodecylbenzene sulfonates and mixtures thereof. Additionally, the surfactant may help dissolve the dye.

Furthermore, the analyte detection systems of the present invention may further compromise a means for reducing impurity content and/or iron content in the water of the toilet tank or bowl. Reducing impurities such as iron content as well as other ions is beneficial since the analyte assay method may be hindered by the presence of iron or other impurities in the toilet bowl water. An ion exchange resin filter system is commonly used to reduce impurities, iron and other ionic content from water, especially in water softeners. Some examples of ion exchange resins filters utilized especially to remove iron include Dowex G-26, Dowex 21K XLT, Dowex MAC-3 and MTO-Amberlite, all manufactured by Supelco. Other methods for removing impurities and iron from the water are known in the art and may include electrolytic ion exchange systems.

In these water treatment systems, untreated water is filtered and passed through a tank filled with small polystyrene beads, also known as ion exchange resin. These beads carry a negative charge and are covered with sodium ions. As the untreated water is passed through the resin, the negatively charged beads attract positively charged ions from the water. The sodium on the resin is then replaced or swapped with natural salts such as calcium, magnesium and iron thereby producing treated water with a reduced ion content. Some of the sodium remains in the treated water, but this does not hinder the assay. This water allows analyte assays to be performed in the toilet bowl with less interference from contaminants, thus creating a better environment for assay testing. Eventually, the resin becomes saturated with ions and must be regenerated by passing a stream of sodium solution over the beads to wash away and displace all of the ions that have built up on the resin. Alternatively, the resin and filter may be exchanged.

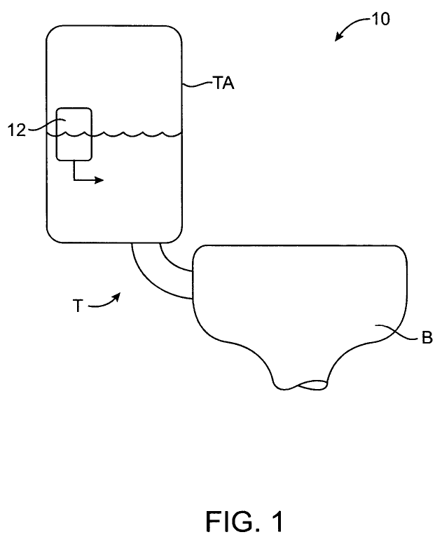

Referring now to FIG. 1, first exemplary system 10 constructed in accordance with the principles of the present invention comprises a dispenser 12 which is mountable within the tank TA of a toilet T. Dispenser 12 is positioned so that it is at least partially covered by water when the tank TA is replenished after flushing. The immersion of the dispenser 12 in water will automatically cause the release of an amount of dye reagent into the water, as indicated by the arrow. The release can be by simple dissolution, by mechanical release (e.g. opening and closing of a float valve or similar mechanical mechanism), by electronic sensing of the water level and a motorized or other powered release of dye from the dispenser 12, and the like. The released dye reagent will remain in the water in the toilet tank until the toilet is next flushed, when the water will enter the toilet bowl B, where it will remain until the use of the toilet by a patient. In other embodiments, the dispenser 12 may have two compartments, one for the dye detector and the other for an oxidizer. In still other embodiments, dispenser 12 may have multiple compartments to hold other detectors for indicating the presence of other substances such as sugar or certain hormones.

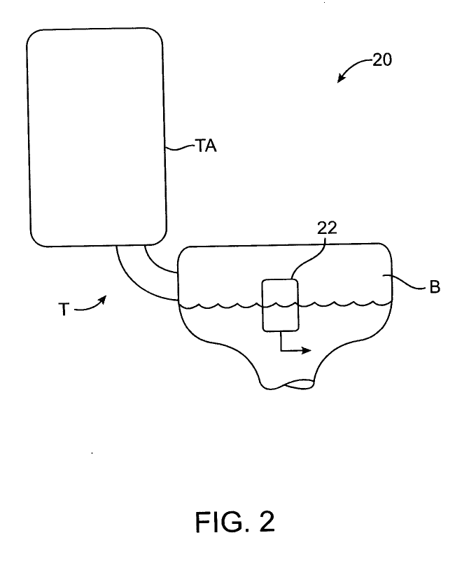

An alternative system 20 as shown in FIG. 2, relies on a dispenser 22 present in the bowl B of the toilet T. The dispenser 22 may take generally the same forms as described above with respect to sensor 12 in FIG. 1, except that the release will be in response to changes in water level within the toilet bowl B. Additionally, the dispenser 22 may also have multiple compartments containing an oxidizer and/or other detectors as discussed previously.

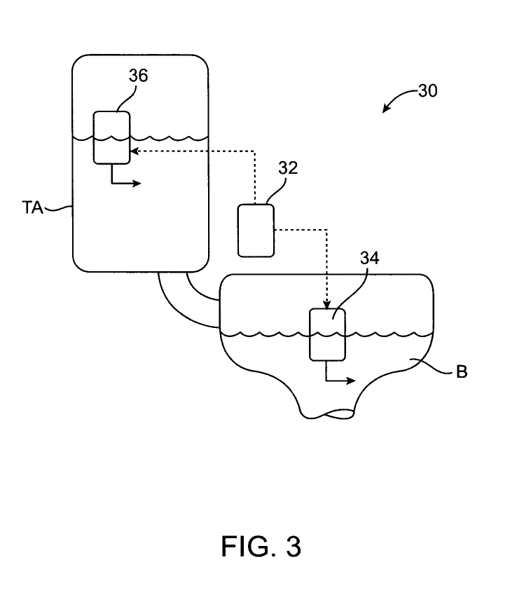

Additional systems 30 according to the present invention are illustrated in FIG. 3. Such systems 30 will comprise an electronic controller 32 which controls operation of a first dispenser 34 located in the toilet bowl B and/or a second dispenser 36 located in the toilet tank TA. New electronic control 32 may comprise one or more sensors which detect a toilet use, such as a water level change, sitting on the seat of the toilet, proximity of the patient to the toilet, the presence of fecal matter and/or urine in the water of the toilet bowl, or the like. In response to one or more of these sensed conditions, the control may cause the first dispenser 34 and/or the second dispenser 36 to release one or more components of the dye reagent into the water in the bowl and/or tank, respectively. Both dispensers may also comprise multiple compartments that additionally allow an oxidizer and other detectors to be dispensed as discussed above.

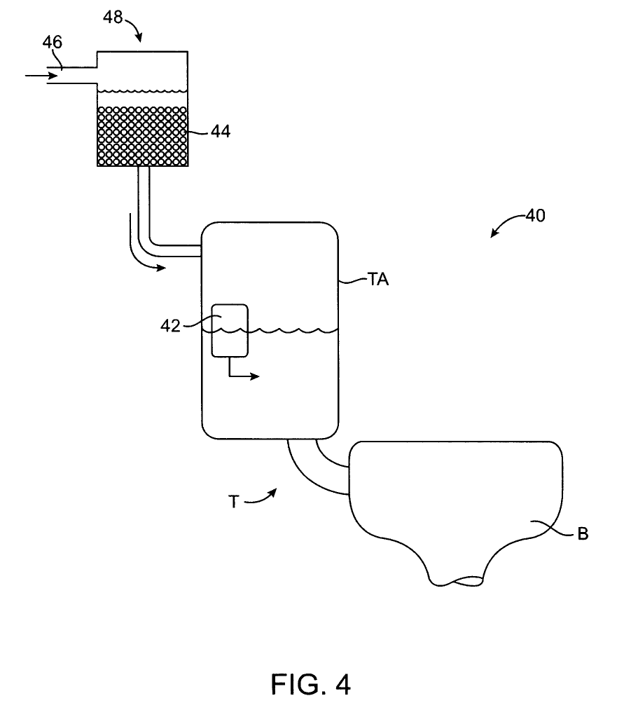

Referring now to FIG. 4, another exemplary system 40 constructed in accordance with the principles of the present invention comprises an ion exchange filter reservoir 48 which contains resin 44. Untreated water enters inlet 46 of the reservoir 48 and passes through the resin 44 and is filtered. Some impurities are removed by filtering, while the resin removes iron and other ions from the water which then flows to the toilet tank, TA, where it is stored until the toilet T is flushed. The ion exchange filter reservoir 48 may optionally be located further downstream in the system 40, for example in between the toilet tank TA and the bowl, B, or the ion exchange filter reservoir 48 may be located in the toilet bowl, B.

The system further comprises a dispenser 42 which is mountable within the tank TA of a toilet T. Dispenser 42 is positioned so that it is at least partially covered by water when the tank TA is replenished after flushing. The immersion of the dispenser 42 in water will automatically cause the release of an amount of dye reagent and surfactant into the water, as indicated by the arrow. The release can be by simple dissolution, by mechanical release (e.g. opening and closing of a float valve or similar mechanical mechanism), by electronic sensing of the water level and a motorized or other powered release of dye from the dispenser 42, and the like. The released dye reagent and surfactant will remain in the water in the toilet tank until the toilet is next flushed, when the water will enter the toilet bowl B, where it will remain until the use of the toilet by a patient. Optionally, the surfactant and the dye may be dispensed from separate dispensers. Furthermore, as discussed above the dispenser 42 may have additional compartments to permit dispensing of an oxidizer and other detectors.

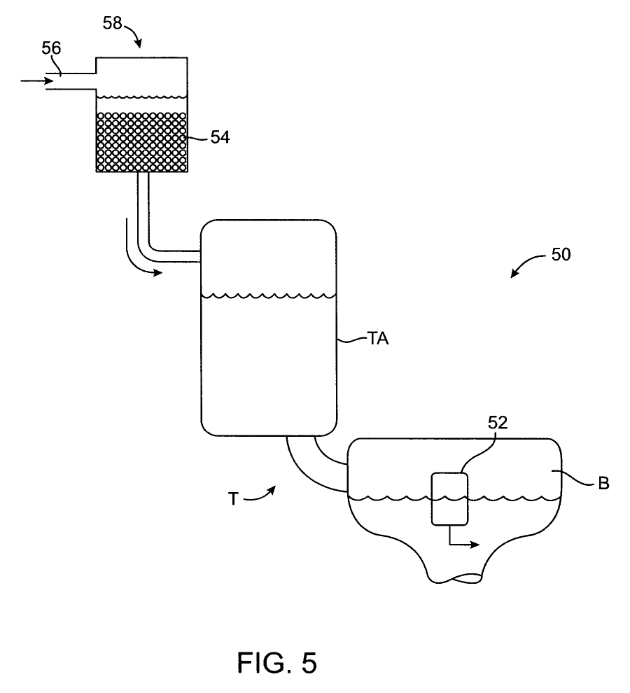

An alternative system 50 as shown in FIG. 5. This system relies on a dispenser 52 present in the bowl B of the toilet T. The dispenser 52 may take generally the same forms as described above with respect to sensor 42 in FIG. 4, except that the release will be in response to changes in water level within the toilet bowl B. Additionally, the system 50 comprises an ion exchange filter reservoir 58. Untreated water enters the reservoir 58 at inlet 56. This water passes over resin 54, is filtered and removes iron and other impurities and ions from the water. The water then flows into the toilet tank TA. The ion exchange filter reservoir and 58 also may take generally the same form as described above with respect to the ion exchange filter reservoir 48 in FIG. 4.

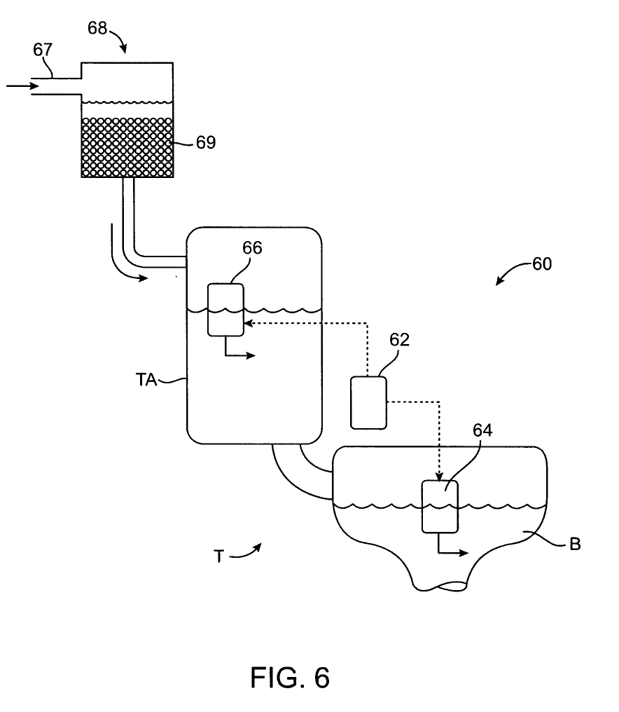

Additional systems 60 according to the present invention are illustrated in FIG. 6. Such systems 60 will comprise an electronic controller 62 which controls operation of a first dispenser 64 located in the toilet bowl B and/or a second dispenser 66 located in the toilet tank TA. The new electronic controller 62 may comprise one or more sensors which detect a toilet use, such as a water level change, sitting on the seat of the toilet, proximity of the patient to the toilet, the presence of fecal matter and/or urine in the water of the toilet bowl, or the like. In response to one or more of these sensed conditions, the control may cause the first dispenser 64 and/or the second dispenser 66 to release one or more components of the dye reagent and surfactant into the water in the bowl and/or tank, respectively. Aspects of the dispensers 64 and/or 66 also may take similar forms as discussed previously with respect to dispensers 34 and 36 in FIG. 3 above. Also aspects of the ion exchange filter reservoir 68 are similar to those described above. Untreated water enters the inlet 67 of the reservoir 68. The water then passes over resin 69 and is filtered, which removes impurities, iron and other ions and then flows to the tank TA of the toilet T. Aspects of the ion exchange filter reservoir 68 are generally in the same form as previously described above for the ion exchange filter reservoirs 48 and 58 in FIGS. 4 and 5, respectively.

The invention has been described above in conjunction with particular embodiments. One skilled in the art, however, will appreciate that there are many alternatives, modifications, and variations of the embodiments which will fall within the scope of the claims below. The present invention is intended to embrace all such alternatives, modifications, and variations within these claims.

What is claimed is:

1. A method for detecting an analyte in water in a bowl of a flush toilet, said method comprising:
maintaining water having a reduced impurity content in the bowl; and
dispersing an amount of a dye reagent in the water in the presence of a surfactant in an amount sufficient to release analyte from stool, wherein the dye reagent produces an observable signal in the presence of the analyte and an oxidizing agent.

2. A method as in claim 1, wherein the impurity comprises iron.

3. A method as in claim 1, wherein the dye reagent is maintained with an anti-oxidant and both are dispersed automatically in response to a use of the toilet.

4. A method as in claim 1, wherein the dye reagent is maintained separately from an oxidant and both are dispersed in response to use of the toilet.

5. A method as in claim 4, wherein the dye reagent and oxidant are dispersed in response to sitting on a seat of the toilet.

6. A method as in claim 1, wherein the dye and the surfactant are dispersed together into the water.

7. A method as in claim 1, wherein the water is treated to remove impurities before being released into the toilet bowl.

8. A method as in claim 7, wherein the impurity comprises iron.

9. A method as in claim 6, wherein the water is treated to remove impurities before being released into a water tank of the flush toilet.

10. A method as in claim 9, wherein the impurity comprises iron.

11. A method as in claim 8, wherein an ion exchange resin removes iron from the water.

12. A method as in claim 11, wherein the ion exchange resin is selected from the group consisting of Dowex G-26, Dowex 21K XLT, Dowex MAC-3 and MTO-Amberlite.

13. A method as in claim 8, wherein an electrolytic ion exchange system removes iron from the water.

14. A method as in claim 1, wherein dispersing comprises releasing the combination of the dye reagent, the surfactant and an anti-oxidant together, and an oxidant separately, into the water in a water tank of the toilet, wherein the water and all reagents are mixed as the water flows into the toilet bowl after flushing the toilet.

15. A method as in claim 14, wherein releasing comprises dropping measured amounts of the reagents into the tank in response to flushing the toilet.

16. A method as in claim 15, wherein the measured amounts comprises a liquid.

17. A method as in claim 15, wherein the measured amounts comprises a gel.

18. A method as in claim 15, wherein the measured amounts comprises a powder.

19. A method as in claim 14, wherein releasing comprises dissolving an amount of solid dye reagent disposed in the tank.

20. A method as in claim 1, wherein dispersing comprises releasing the dye reagent directly into the water in the bowl of the toilet.

21. A method as in claim 20, wherein releasing comprises dropping a measured amount of the dye reagent into the bowl in response to flushing the toilet.

22. A method as in claim 21, wherein the measured amount comprises a liquid.

23. A method as in claim 21, wherein the measured amount comprises a gel.

24. A method as in claim 24, wherein the measured amount comprises a powder.

25. A method as in claim 20, wherein releasing comprises dissolving an amount of solid dye reagent disposed in the toilet bowl.

26. A method as in claim 1, wherein the dye reagent produces a color change in the water in the presence of the analyte.

27. A method as in claim 26, wherein the dye reagent produces a color change in water in the presence of blood.

28. A method as in claim 27, wherein the dye reagent produces an observable color change at local blood concentrations in the water of 0.2 ppm and above.

29. A method as in claim 28, wherein the dye reagent comprises an oxidizer and a dye, wherein the oxidizer oxidizes the dye to produce the color change in the presence of a peroxidase from blood hemoglobin.

30. A method as in claim 29, wherein the dye is selected from the group consisting of 3,3′,5,5′-tetramethylbenzidine, gum guaiac, potassium guaicosulfonate, phenolphthalin, 3,3′-dimethylbenzidine, o-toluidine, 4,4′-diaminobiphenyl, and the oxidizer is selected from the group consisting of alkali metal perborates, OXONE, and hydrogen peroxide.

31. A method as in claim 1, further comprising adding an analyte-containing control substance into the water to confirm that the observable signal is produced.

32. A method as in claim 1, wherein the surfactant is selected from the group consisting of fatty alkanolamides, isopropyl amine branched dodecylbenzene sulfonates, calcium alkylbenzene sulfonates, hydrogenated cocamidopropyl betaines, anionic/lignosulfonates blends, linear alkylbenzene sulfonic acids, and linear sodium dodecylbenzene sulfonates.

33. A method as in claim 1, further comprising dispersing a second detector in the water, wherein the detector produces an signal in the presence of a second analyte.

34. A system for automatically dispensing a dye reagent into water in a bowl of a flush toilet, said system comprising:
a first reservoir holding a dye and an anti-oxidant, wherein the dye produces an observable signal in the presence of an analyte and an oxidizer;
a second reservoir holding a surfactant; and
means for dispensing an amount of the dye reagent into the water in the toilet bowl in response to a use of the toilet.

35. A system as in claim 34, wherein the dispensing means releases the dye reagent into the water in the toilet bowl or in a water tank of the toilet, wherein the water and dye reagent are mixed in the toilet bowl.

36. A system as in claim 35, wherein the dispensing means drops a measured amount of the dye reagent into the water in response to flushing the toilet.

37. A system as in claim 36, wherein the measured amount is a liquid.

38. A system as in claim 36, wherein the measured amount is a gel.

39. A system as in claim 36, wherein the measured amount is a powder.

40. A system as in claim 35, wherein the dispensing means dissolves an amount of solid dye reagent disposed in the tank.

41. A system as in claim 34, wherein the dispensing means dispenses the dye and the oxidizer separately into the water.

42. A system as in claim 34, wherein the dispensing means maintains and dispenses the dye and the oxidizer together into the water.

43. A system as in claim 34, wherein the dye reagent produces a color change in the water in the presence of the analyte.

44. A system as in claim 35, wherein the dye reagent produces a color change in water in the presence of blood.

45. A system as in claim 36, wherein the dye reagent produces an observable color change at local blood concentrations in the water of 0.2 ppm and above.

46. A system as in claim 37, wherein the dye reagent comprises an oxidizer and a dye, wherein the oxidizer oxidizes the dye to produce the color change in the presence of a peroxidase from blood hemoglobin.

47. A system as in claim 46, wherein the dye is selected from the group consisting of 3,3′,5,5′-tetramethylbenzidine, gum guaiac, potassium guaicosulfonate, phenolphthalin, 3,3′-dimethylbenzidine, o-toluidine, 4,4′-diaminobiphenyl, and the oxidizer is selected from the group consisting of alkali metal perborates, OXONE, and hydrogen peroxide.

48. A system as in claim 34, further comprising a third reservoir holding a second detector, wherein the detector produces a signal in the presence of a second analyte.

49. A system for automatically dispensing a dye reagent into water in a bowl of a flush toilet, said system comprising:
a first reservoir holding a dye and an anti-oxidant, wherein the dye produces an observable signal in the presence of an analyte and an oxidizer;
a second reservoir holding a surfactant;
means for dispensing an amount of the dye reagent into the water in the toilet bowl in response to a use of the toilet bowl; and
means for reducing impurity content in the water of the toilet bowl.

50. A system as in claim 47, wherein the impurity is iron.

51. A system as in claim 49, wherein the dispensing means releases the dye reagent into the water in the toilet bowl or in a water tank of the toilet, wherein the water and dye reagent are mixed in the toilet bowl.

52. A system as in claim 47, wherein the dispensing means drops a measured amount of the dye reagent into the water in response to flushing the toilet.

53. A system as in claim 52, wherein the measured amount is a liquid.

54. A system as in claim 52, wherein the measured amount is a gel.

55. A system as in claim 52, wherein the measured amount is a powder.

56. A system as in claim 51, wherein the dispensing means dissolves an amount of solid dye reagent disposed in the tank.

57. A system as in claim 49, wherein the dispensing means dispenses the dye and oxidizer separately into the water.

58. A system as in claim 49, wherein the dispensing means maintains and dispenses the dye and the oxidizer together into the water.

59. A system as in claim 49, wherein the dye reagent produces a color change in the water in the presence of the analyte.

60. A system as in claim 51, wherein the dye reagent produces a color change in the water in the presence of blood.

61. A system as in claim 52, wherein the dye reagent produces an observable color change at local blood concentrations in the water of 0.2 ppm and above.

62. A system as in claim 52, wherein the dye reagent comprises an oxidizer and a dye, wherein the oxidizer oxidizes the dye to produces the color change in the presence of a peroxidase from blood hemoglobin.

63. A system as in claim 62, wherein the dye is selected from the group consisting of 3,3′,5,5′-tetramethylbenzidine, gum guaiac, potassium guaicosulfonate, phenolphthalin, 3,3′-dimethylbenzidine, o-toluidine, 4,4′-diaminobiphenyl, and the oxidizer is selected from the group consisting of alkali metal perborates, OXONE, and hydrogen peroxide.

64. A system as in claim 49, wherein the means for reducing impurity content treats the water before being released into the bowl of a flush toilet.

65. A system as in claim 49, wherein the means for reducing impurity content treats the water before being released into a water tank of the flush toilet.

66. A system as in claim 49, wherein the means for reducing iron content comprises an ion exchange resin.

67. A system as in claim 66, wherein the ion exchange resin is selected from the group consisting of Dowex G-26, Dowex 21K XLT, Dowex MAC-3 and MTO-Amberlite.

68. A system as in claim 49, wherein the means for reducing iron content comprises an electrolytic ion exchange system.

69. A system as in claim 49, further comprising a third reservoir holding a second detector, wherein the detector produces an signal in the presence of a second analyte.
